(19) United States
(12) Patent Application Publication
(10) Pub. No.: US 2022/0196659 A1
(43) Pub. Date: Jun. 23, 2022

(54) METHOD FOR DETERMINING EFFICACY

(71) Applicant: LDN Pharma Limited, London, England (GB)

(72) Inventor: Wai Liu, London (GB)

(21) Appl. No.: 17/435,911

(22) PCT Filed: Mar. 6, 2020
(86) PCT No.: PCT/EP2020/056113

§ 371 Date: Sep. 2, 2021

(30) Foreign Application Priority Data
Mar. 6, 2019 (EP) 19161136.7

(51) Int. Cl. G01N 33/573; G01N 33/561; G01N 33/574

(52) U.S. Cl. CPC G01N 33/573; G01N 2800/52; G01N 33/574; G01N 33/561

(57) ABSTRACT

The invention relates to a method for monitoring the treatment of a subject undergoing therapy with an active that is naltrexone or a metabolite or analogue thereof, comprising:
a. measuring the level of pERK in a sample obtained from the subject undergoing treatment;
b. comparing the level of pERK with a reference,
   wherein if the pERK level is increased compared to the reference, then the active is being administered at an effective level.

### FIELD OF THE INVENTION

The invention relates to methods of monitoring treatment of a subject by monitoring the expression level of a particular biomarker.

### BACKGROUND OF THE INVENTION

The success of many cancer therapies is based upon co-administration of an active compound targeting the cancer alongside adjuvant-type molecules. Without any independent therapeutic utility, adjuvants are responsible for priming the immune system of a subject such that the active compound targeting the cancer can achieve maximum therapeutic effect.

As adjuvants typically modulate the immune response of a patient, they are used most commonly in conjunction with cancer vaccines or biologics such as humanized therapeutic antibodies. They act either to enhance the immune system of a patient to increase the production of antibodies in response to challenge with a cancer vaccine, or by supressing or lowering the immunogenicity of the patient towards a foreign therapeutic antibody. Thus, adjuvants play an important role in driving immune cancer therapies towards a successful therapeutic outcome.

Often, adjuvants will be combined with more traditional cancer therapies such as radiotherapy or chemotherapy resulting in a synergistic treatment whereby the efficacy of the therapy is significantly increased due to the presence of the adjuvant. Therefore, the amount of the traditional cancer therapies used can be reduced, which is beneficial as many cancer therapies are associated with negative side-effects when administered in high doses.

Due to the many varying factors that affect the effect the therapies are having on the body, such as the age, gender, height and weight of a patient as well as environmental factors, it is difficult to know if therapeutics and adjuvants are being administered at an effective level. Furthermore, traditional cancer therapies frequently result in negative side-effects experienced by the patient, which can be exacerbated by non-optimum doses, which again can vary in different patients.

Thus, there is a need to develop new treatment regimens that enable monitoring of the effect that the therapies are having on individual patients to ensure that patients are receiving optimum treatment dosages. If it is apparent from the monitoring that this is not the case, then the amount of the therapeutic drug administered can be altered accordingly to help the treatment to be as effective as possible whilst minimising the negative side-effects of the therapies.

### SUMMARY OF THE INVENTION

It is known that co-administration of naltrexone alongside a chemotherapeutic agent, results in a reduction in cancer cell growth compared to administering a chemotherapeutic agent alone. However, it would be beneficial if there was a way to quantify the effectiveness of the dose given, to ensure that the optimal treatment is given.

It has been previously reported by the present inventors that low dose naltrexone has advantages during medical treatment over previously used higher dosage regimes. It has now been found by the present inventors that treatment using a low dose of an active that is naltrexone or a metabolite or analogue thereof increases the expression of pERK in a subject. pERK expression drives the proliferation of active T cells (D'Souza et al., J Immunol., 2008, 181(11): 7617-7629.) thus it promotes the T cell function of providing an immunological response that targets destroying cells that are infected or cancerous. However, administration of higher doses of an active, such as naltrexone or a metabolite or analogue thereof, has been found to have the opposite effect and decreases the expression of pERK in a subject. Consequently, too much of the active does not help to increase the number of active T cells that help the body to destroy cancerous or infected cells. Therefore, it is beneficial to subjects receiving treatment as described herein to be administered an active that is naltrexone or a metabolite or analogue thereof at a level that will raise the pERK expression of the subject to improve their immunological response (i.e. promote T cell proliferation). Using the method of the present invention, the levels of pERK expression can be monitored to determine if the active is being administered at the desired low dose and at an effective level to ensure that the pERK expression is increased.

According to a first aspect of the invention, there is provided a method for monitoring the treatment of a subject undergoing therapy with an active that is naltrexone or a metabolite or analogue thereof, comprising:
a. measuring the level of pERK in a sample obtained from the subject undergoing treatment;
b. comparing the level of pERK with a reference,
   wherein if the pERK level is increased compared to the reference, then the active is being administered at an effective level.

### DESCRIPTION OF THE DRAWINGS

The invention is described with reference to the accompanying drawings, wherein:

FIG. 1a-e: Shows the pERK, p21 and BAX expression levels at 0 h and 48 h produced by tablets containing (a) 1.5 mg, (b) 3.0 mg, (c) 4.5 mg, (d) 6.0 mg, and (e) 10 mg.

FIG. 2: Shows the Western blotting of the PBMC samples after naltrexone (NTX) administration at different amounts.

FIG. 3: Shows the pERK, p21 and BAX expression levels produced by tablets containing 1.5 mg, 3.0 mg, 4.5 mg, 6.0 mg, and 10 mg.

FIG. 4: Shows the negative correlation between pERK expression levels and Cₘₐₓ for naltrexone (left) and 6-β-naltrexol (right).

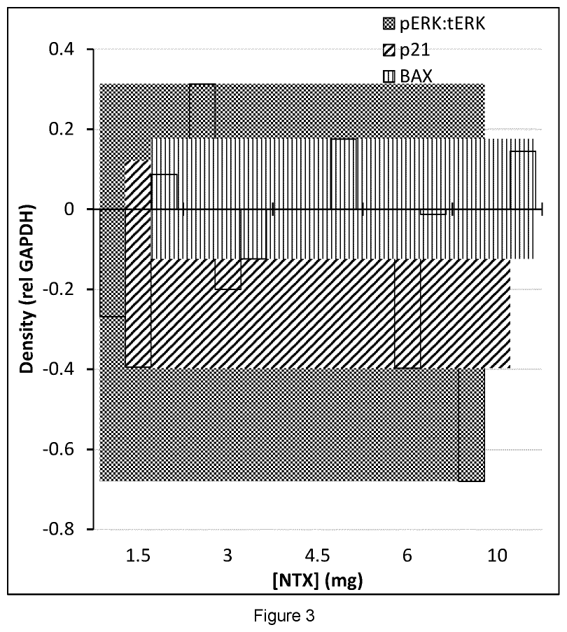

FIG. 5: Shows the results from Example 2; the CD69 expression of PBMCs cultured with LDN, NTX or DMSO.

### DETAILED DESCRIPTION OF THE INVENTION

It has been found by the present inventors that low dose naltrexone (LDN) has beneficial “priming” effects on the immune system. Thus, LDN is to be used to prime the cells of the immune system prior to treatment with further drugs/therapeutic options.

Priming with LDN as part of a first treatment phase prior to administration of further drugs has been shown by the present inventors in earlier applications that it results in greater cell kill than no priming with LDN phase and continuous LDN administration. However, there is currently no simple method of determining if an appropriate amount of LDN has been administered and thus if the priming effects are present.

The invention is based on the finding that the administration of a low dose of an active, naltrexone or a metabolite or analogue thereof, increases the expression of the biomarker phosphorylated ERK (pERK) in a subject. This is exemplified by the compound naltrexone. Certain levels of pERK expression appear to be found in patients receiving an effective low dose. Thus, by measuring the level of pERK expression in a subject, whilst they are undergoing therapy with naltrexone or a metabolite or analogue thereof, it enables the physician to monitor if the drug is being administered at the desired low dose and at an effective level. Therefore, the present inventors have devised an in vitro method for monitoring the treatment by measuring the expression levels of pERK, which in turn allows the amount of the active naltrexone or a metabolite or analogue thereof being administered to be altered to maximise efficacy and safety.

Active agents such as naltrexone or a metabolite or an analogue thereof when administered to a subject undergoing therapy enhances the cytotoxicity or cytostatic activity of the anti-cancer agent the subject is being treated with. Without wishing to be bound by theory, the increase in the expression levels of pERK is an indication that the cell is reacting to the effect of the active naltrexone or a metabolite or an analogue thereof and therefore shows that the active is being administered at an effective low dose. On the other hand, administration of too much or too little of an active agent causes a decrease in the pERK expression which may result in the active having a reduced adjuvant effect on the anti-cancer agent. CD3+ cells are immunological cells that play an important role in the body to fight against disease. Activation of CD3+ cells in turn increase the proliferation of T cells, which are key in the body's defence against disease. Hence, the immune system is effectively boosted. Furthermore, the increase in activity of these cells increases their overall cytotoxicity and subsequently the cytotoxicity of the therapy regimen, which includes administering a further treatment drug. pERK is therefore a useful marker for identifying if the amount of the active being administered is at the desired low dose and at an effective level to enhance the cytotoxicity or cytostatic activity of the further treatment drug, which is subsequently administered if the active is at the effective level.

In certain embodiments, the active is provided in an amount sufficient to increase the expression level of pERK above the normal basal levels expected in a subject. For example, the active may be administered in an amount sufficient to increase the level of expression of pERK by at least 10%, preferably at least 20%, more preferably at least 30%, even more preferably at least 40%, even more preferably at least 50%, even more preferably at least 60%, compared to the normal basal levels expected in the subject. The “normal” basal level may be determined by measuring the level of expression of pERK in a sample obtained from the subject prior to the administration of the active or maybe a reference control, established by measuring the basal level of expression in a normal healthy subject.

It has also been found that there is an optimum range in which the pERK expression levels are increased to when the active is being administered at its most effective dose. Therefore, in certain embodiments, whilst the active is administered in an amount sufficient to increase the expression level of pERK above the normal basal levels expected in a subject as described above, the amount of active administered increases the expression level of pERK by at most 90%, or preferably by at most 85%, or more preferably by at most 80%, or even more preferably by at most 75%, even more preferably by at most 70%, compared to the normal basal levels expected in the subject.

Suitably the active is administered in an amount sufficient to increase the expression level of pERK to between 15% and 90%, preferably between 20% and 85%, more preferably between 25% and 80%, even more preferably between 35% and 75%, even more preferably between 40% and 70%, even more preferably between 45% and 65%, even more preferably between 50% and 60% of the normal basal levels expected in the subject.

The level of expression of pERK can be measured in the population of cells using any number of analytical methods available to the skilled person, including, but not limited to, gel electrophoresis and Western blot analysis, 2D-PAGE, column chromatography, ribosome profiling or mass spectrometry. The increased level of expression can be determined by comparing the level of expression of the biomarker from before and after administration of the active that increases the level of expression of pERK.

In certain embodiments, the biological sample obtained from the subject for use in the method is blood, plasma, serum, lymph fluid, a tissue, or cells derived from a tissue sample, preferably blood. Preferably the sample comprises white blood cells, such as peripheral blood mononuclear cells (PBMCs), preferably CD3+ cells. Conventional techniques for obtaining any of the above biological samples from a subject are well known to the person skilled in the art. Being able to use a blood sample provides a convenient and easy platform of measuring the gene expression to then in turn establish if the priming of the immune system has occurred.

The “reference” value for use in the method can be the level of expression of pERK determined from a biological sample obtained from a healthy subject. As used herein, a “healthy subject” refers to a subject who is not suffering from cancer or a CNS disorder. The reference value may be determined by measuring the level of expression of pERK in the sample obtained from a healthy individual at the time the method of the invention is performed. Alternatively, the reference value may be a pre-determined value from a prior measurement of the level of expression of pERK in an equivalent sample obtained from a healthy individual. When monitoring the treatment of the active that increases the expression of pERK, the reference value may be that derived from a healthy individual, or the reference value may be the pERK concentration measured in the sample previously obtained from the subject, i.e. the reference value may be the level of expression of pERK in a sample obtained from the subject prior to administration of the active.

Comparing the pERK level of the subject to a reference is used to determine if the amount of active being administered is at a desired low dose and at an effective level. When the pERK level is increased to the optimum levels compared to the reference as described herein, then the active is being administered at a desired low dose and at an effective level. On the contrary, when the pERK level is at the same level or below the reference, then the active is not being administered at an effective level. Therefore, the amount of active administered in the treatment regime can be adjusted accordingly, i.e. the amount of active administered is increased or decreased accordingly if it is not being administered at an effective level.

Suitably the level of pERK is measured at least 24 hours after administration of the active, more suitably at least 48 hours after administration of the active.

Suitably, the active is to be administered at a low dose i.e. in an amount effective to increase the blood plasma concentration of the active to at least 0.34 ng/ml, or at least 3.4 ng/ml, or at least 34 ng/ml, or at least 340 ng/ml. In certain embodiments, the active is to be administered in an amount effective to increase the blood plasma concentration of the active to within the range of 0.3 ng/ml to 3,400 ng/ml, preferably to within the range of from 34 ng/ml to 3,400 ng/ml more preferably 340 ng/ml to 3,400 ng/ml. The amount effective to achieve such an amount can be determined using any number of conventional techniques known to the person skilled in the art. For example, the skilled person could perform mass spectrometry on a blood plasma sample obtained from the subject in order to determine the increase in the concentration of the active within the sample after administration of an amount of the active. The effective amount is the amount determined to bring about the desired increase in blood plasma concentration.

In one embodiment, the low dose, i.e. the effective amount, per day of the active employed in the therapy may be from about 0.01 mg to up to 10 mg, preferably from about 0.1 mg to about 8 mg, most preferably from about 1 to about 6 mg of the active; e.g. about 0.01 mg, about 0.05 mg, about 0.1 mg, about 0.3 mg, about 0.5 mg, about 1 mg, about 2 mg, about 3 mg, about 5 mg, about 10 mg, about 20 mg, about 30 mg, about 40 mg or about 50 mg of the active employed per day. In certain embodiments the effective amount per day of the active employed is no more than 4.5 mg, such as from 2 mg to 4.5 mg or from 3 mg to 4.5 mg, preferably 3 mg to 4 mg.

As used herein, the terms “treating” and “treatment” and “to treat” and “therapy” refer to both 1) therapeutic measures that cure, slow down, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented.

As used herein “naltrexone” refers to morphinan-6-one,17-(cyclopropylmethyl)-4,5-epoxy-3,14-dihydroxy-(5+), and pharmaceutically acceptable salts, solvates, hydrates, stereoisomers, clathrates and prodrugs thereof. The use of naloxone, a structural analogue of naltrexone, is within the purview of the invention and is encompassed within the term “analogue” used in the description and the claims. Similarly, methylnaltrexone is also envisaged as a suitable analogue for use in all aspects of the invention.

6-β-naltrexol is a major active metabolite of naltrexone, which is encompassed with the term “metabolite” used in the description and claims. As used herein “6-β-naltrexol” refers to 17-(Cyclopropylmethyl)-4,5-epoxymorphinan-3,6beta,14-triol and pharmaceutically acceptable salts, solvates, hydrates, stereoisomers, clathrates and prodrugs thereof. The term 6-β-naltrexol also encompasses functionally equivalent analogues thereof and metabolites that retain functional equivalence with respect to the novel uses of 6-β-naltrexol embodied within the invention. The preferred form of naltrexone or a metabolite or an analogue thereof is as the hydrochloride salt form.

As used herein, “cytotoxicity” refers to the quality of an agent being toxic to cells. Cytotoxicity may therefore refer to the ability of an agent to induce cell death upon coming into contact with a cell. The cytotoxic mechanism leading to cell death may be due to necrosis or programmed cell death (apoptosis). Cytotoxicity may be measured in a population of cells using any number of cell viability assays, or by using antibodies specific for protein factors activated upon the initiation of apoptosis.

As used herein, “cytostasis” refers to the inhibition of cell growth and multiplication. Thus, a cytostatic agent may refer to an agent that inhibits the proliferation or growth of a cell, perhaps without causing cytotoxicity. An agent that causes cytostasis can be determined by measuring the DNA content of individual cells within a population of cells. A population of cells undergoing proliferation will have subpopulations of cells with varying levels of DNA content. The DNA content within the cell will be dependent on the phase of the cell cycle within which the cell resides. Where an agent causes cytostasis, the balance of the population of cells within each phase of the cell cycle will be abnormal. For example, if cytostasis occurs in the S phase or G₂ phase, an abnormal number of cells will contain twice the content of DNA normally observed in a somatic cell. Conversely, if cytostasis occurs during the G₀ or G₁ phase, an abnormal number of cells will contain the amount of DNA typically observed in a somatic cell.

As used herein, the term “subject” refers to any animal (for example, a mammal), including, but not limited to, humans, non-human primates, canines, felines, rodents, and the like. Typically, the terms “subject” and “patient” are used interchangeably herein in reference to a human subject.

The subject may be being treated for a number of disorders, as the priming effect of the LDN boosts the immune system, it can be beneficial for use in an array of treatments targeting different diseases. One such example is in patients that have been immunocompromised due to a disease itself (for example HIV/AIDS) or the treatment associated with a disease (for example chemotherapy during cancer treatment). “Immunodeficiency” or “immunocompromised” is a state in which the immune system is weakened or impaired thereby reducing its ability to fight infectious disease. Preferably, the treatment is intended either as, or as part of, a regimen for treating a cancer, preferably in the treatment of breast, lung, melanoma, colon or glioma cancer.

As used herein, the term “HIV” refers to “human immunodeficiency virus”, which is a virus that damages the cells in your immune system and weakens your ability to fight everyday infections and disease. “AIDS” refers to “acquired immune deficiency syndrome”, which describes a number of potentially life-threatening infections and illnesses that happen when your immune system has been severely damaged by the HIV virus.

Typically, HIV is treated with antiretroviral medicines. As such, after priming with LDN antiretroviral therapy may be administered. Antiretroviral therapy is typically prescribed using three different drug molecules. Examples include nucleoside reverse transcriptase inhibitors (such as abacavir (Ziagen), emtricitabine (Emtriva), lamivudine (Lamivudine RBX, Zefix, Zetlam)), non-nucleoside reverse transcriptase inhibitors (such as delavirdine (Rescriptor), doravirine (Pifeltro), efavirenz (Sustiva)), protease inhibitors (such as atazanavir (Reyataz), darunavir (Prezista), fosamprenavir (Lexiva, Telzir)), entry inhibitors (such as enfuvirtide (Fuzeon), maraviroc (Selzentry)) and integrase inhibitors (such as dolutegravir (Tivicay), elvitegravir (Vitekta), raltegravir (Isentress)).

As used herein, the term “cancer” refers to any mass of tissue that results from excessive cell growth, proliferation and/or survival, either benign (noncancerous) or malignant (cancerous), including pre-cancerous lesions. As used herein, the term “cancer cell” refers to a cell or immortalized cell line derived from cancer.

Typically, cancer treatment includes one or more of chemotherapy, radiation therapy, hormonal therapy or immunotherapy. As such, after priming with LDN cancer treatment may be administered. As used herein “chemotherapy”, “chemotherapeutic agent”, “radiation therapy”, “hormonal therapy” and “immunotherapy” have their conventional meanings in the art. The term “anti-cancer agent” is used synonymously with “chemotherapeutic agent”. Combination therapy may be used whereby multiple known therapies are used.

In certain embodiments, the chemotherapy involves administering an anti-cancer agent selected from the group consisting of PI3-kinase inhibitors, AKT inhibitors, taxanes, antimetabolites, alkylating agents, cell cycle inhibitors, topoisomerase inhibitors and cytotoxic antibodies.

Where the chemotherapeutic agent is a PI3-kinase inhibitor, suitable examples include, but are not limited to, wortmannin, LY294002, demethoxyviridin, IC87114, NVP-BEZ235, BAY 80-6946, BKM120, GDC-0941, GDC-9080; including combinations thereof; and pharmaceutically acceptable salts, solvates, hydrates, stereoisomers, clathrates and prodrugs of any of the above.

Where the anti-cancer agent is an AKT inhibitor, suitable examples include, but are not limited to, MK-2206, GSK690693, perifosine, PHT-427, AT7867, honokiol, PF-04691502; including combinations thereof; and pharmaceutically acceptable salts, solvates, hydrates, stereoisomers, clathrates and prodrugs of any of the above.

Where the anti-cancer agent is a taxane, suitable examples include, but are not limited to, paclitaxel and docetaxel; including combinations thereof; and pharmaceutically acceptable salts, solvates, hydrates, stereoisomers, clathrates and prodrugs of any of the above.

Where the anti-cancer agent is an antimetabolite, suitable examples include, but are not limited to, methotrexate, 5-fluorouracil, capecitabin, cytosinarabinoside (Cytarabin), gemcitabine, 6-thioguanin, pentostatin, azathioprin, 6-mercaptopurin, fludarabin and cladribin; including combinations thereof; and pharmaceutically acceptable salts, solvates, hydrates, stereoisomers, clathrates and prodrugs of any of the above.

Where the anti-cancer agent is an alkylating agent, suitable examples include, but are not limited to, mechlorethamine, cyclophosphamide, ifosfamide, trofosfamide, melphalan (L-sarcolysin), chlorambucil, hexamethylmelamine, thiotepa, busulfan, carmustine (BCNU), streptozocin (streptozotocin), dacarbazine (DTIC; dimethyltriazenoimidazol ecarboxamide) temozolomide and oxaliplatin; including combinations thereof; and pharmaceutically acceptable salts, solvates, hydrates, stereoisomers, clathrates and prodrugs of any of the above.

Where the anti-cancer agent is a cell cycle inhibitor, suitable examples include, but are not limited to, Epothilone, Vincristine, Vinblastine, UCN-01, 17AAG, XL844, CHIR-124, PF-00477736, CEP-3891, Flavopiridol, berberine, P276-00, terameprocol, isoflavone daidzein, BI2536, BI6727, GSK461364, Cyclapolin, ON-01910, NMS-P937, TAK-960, Ispinesib, Monastrol, AZD4877, LY2523355, ARRY-520, MK-0731, SB743921, GSK923295, Lonafarnib, proTAME, Bortezomib, MLN9708, ONX0912, CEP-18770; including combinations thereof; and pharmaceutically acceptable salts, solvates, hydrates, stereoisomers, clathrates and prodrugs of any of the above; particularly suitable examples of cell cycle inhibitors include, but are not limited to, Hespaeradin, ZM447439, VX-680, MLN-8054, PHA-739358, AT-9283, AZD1152, MLN8237, ENMD2076, SU6668; including combinations thereof; and other inhibitors of Aurora kinases; and pharmaceutically acceptable salts, solvates, hydrates, stereoisomers, clathrates and prodrugs of any of the above.

Checkpoint inhibitors are a form of cancer immunotherapy with currently approved inhibitors that target the molecules CTLA4, PD-1, and PD-L1.

In another embodiment, the therapy may also include administering Vitamin D to the subject. Vitamin D and the active may be administered concurrently or simultaneously, sequentially or separately, preferably simultaneously.

As used herein, “vitamin D” refers to vitamin D and any intermediate or product of a metabolic pathway of vitamin D that result in a metabolite that is capable of boosting the cytostatic effect of the active. Metabolite may refer to a vitamin D precursor, which can be incorporated into a vitamin D synthetic pathway occurring naturally within the subject to undergo the therapy of the invention. Alternatively, metabolite may refer to a molecule derived from an anabolic or catabolic process that utilizes vitamin D. Non-limiting examples of vitamin D metabolites include ergocalciferol, cholecalciferol, calcidiol, and calcitriol, 1a-hydroxycholecalciferol, 25-hydroxycholecalciferol, 1a,25-hydroxycholecalciferol, 24,25-hydroxycholecalciferol. An “active” metabolite is a metabolite that can be used in the context of the present invention. Dosage regimes of vitamin D or active metabolites thereof will be well known to the person skilled in the art. The term vitamin D also encompasses pharmaceutically acceptable salts of any of the above. A particularly suitable metabolite of vitamin D for use in the present invention is calcitriol.

As used herein, the terms “concurrently” or “simultaneous”, “sequential” or “separate” mean that administration of the active and the vitamin D product occur as part of the same treatment regimen.

“Simultaneous” administration, as defined herein, includes the administration of the active and the vitamin D product within about 2 hours or about 1 hour or less of each other, even more preferably at the same time.

“Separate” administration, as defined herein, includes the administration of the active and the vitamin D product, more than about 12 hours, or about 8 hours, or about 6 hours or about 4 hours or about 2 hours apart.

“Sequential” administration, as defined herein, includes the administration of the active and the vitamin D product each in multiple aliquots and/or doses and/or on separate occasions. The active may be administered to the patient before or after administration of the vitamin D product. Alternatively, the vitamin D product is continued to be applied to the patient after treatment with the active ceases.

CD69 is a surface marker that becomes expressed as part of the cell's ability to interact with others. This is a sign that the cell is active or becoming active. Thus, CD69 expression is increased in immune cells, such as CD3+ and T cells, when they are activated. Thus, in a second aspect there is provided a method for monitoring the treatment of a subject undergoing therapy with an active that is naltrexone or a metabolite or analogue thereof, comprising:

measuring the CD69 expression in a sample of CD3+ cells obtained from the subject undergoing treatment;

wherein if the CD69 expression is increased compared to a control the active is being administered at an effective level.

The level of expression of CD69 can be measured in the population of cells using any number of analytical methods available to the skilled person, including, but not limited to, gel electrophoresis and Western blot analysis, 2D-PAGE, column chromatography, ribosome profiling or mass spectrometry. All other embodiments described for the first aspect are applicable to the second aspect.

### EXAMPLES

### Example 1

### PD Analysis

There were 5 treatment cohorts with 8 normal volunteers in each cohort. Each volunteer was treated with a single dose of naltrexone as a 1, 3, 4.5, 6 or 10 mg tablet. PBMCs were harvested at 0 and 48 h by ficoll extraction and lysed in cell lysis buffer. Western blotting was then performed on the samples, and pERK, tERK, BAX and p21 measured.

Results

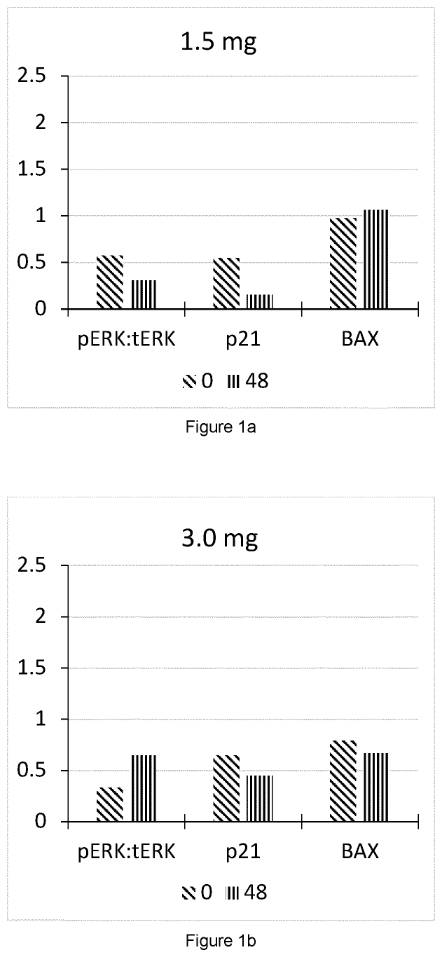

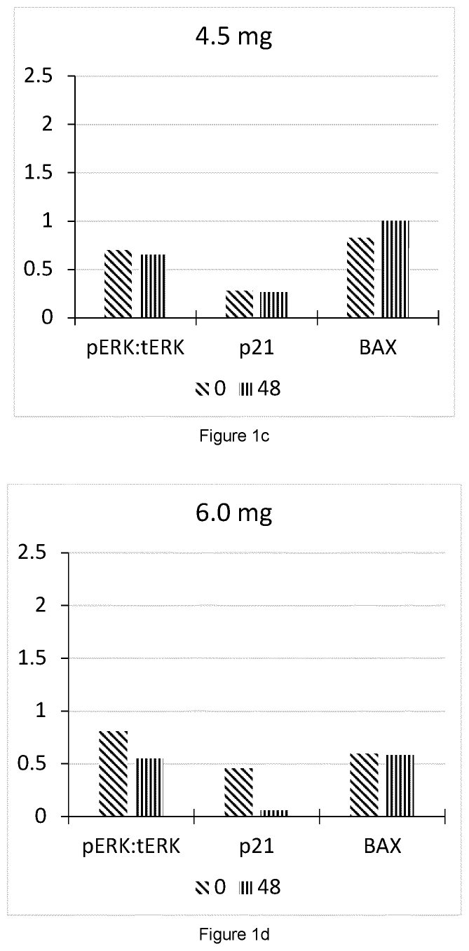

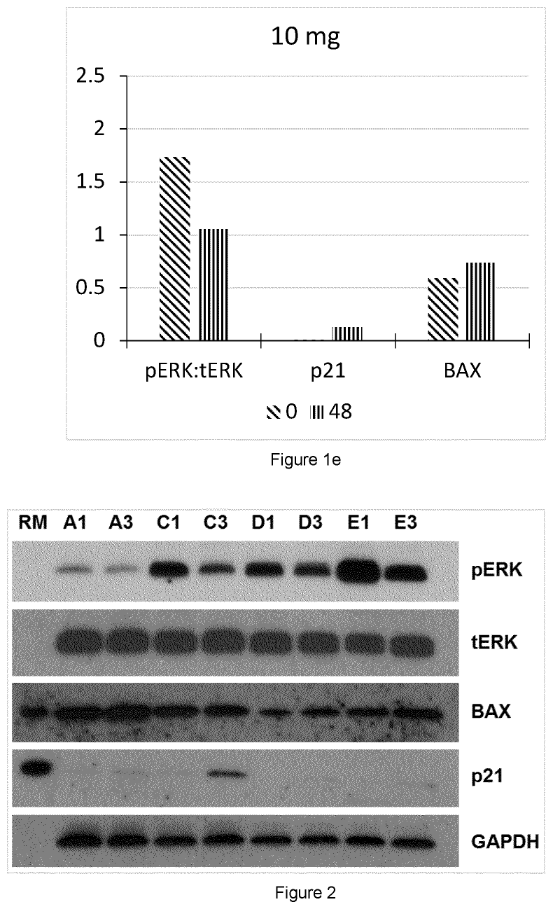

As shown in FIGS. 1a, c-e, the level of pERK expression was decreased in the subjects at 48 h compared to 0 h when administered with a 1, 4.5, 6 or 10 mg tablet. This is also shown by the Western blotting in FIG. 2; the blots for the 48 h measurements A3, C3, D3 and E3 are smaller than their 0 h counterparts A1, C1, D1 and E1. There was found to be a negative correlation between pERK expression and Cₘₐₓ of naltrexone and 6-β-naltrexol, as shown in FIG. 3, suggesting that the higher the Cₘₐₓ of naltrexone and 6-β-naltrexol, the greater the reduction in pERK expression. However, as shown in FIG. 1b, the level of pERK expression was increased in the subjects at 48 h compared to 0 h when administered with a 3 mg tablet i.e. the desired low dose. This shows that there is a clear difference in the pERK expression levels of a subject who has been administered an advantageous low dose of naltrexone compared to a subject who has been administered other doses.

### Example 2

### CD3+ Activation After Administration of LDN

Peripheral-blood mononuclear cells were isolated from whole blood or from the residue product of leucoreduction of whole blood from pathologically healthy donors using Histopaque-1077. The mononuclear fraction was harvested and red blood cell contamination removed by incubation in hypotonic ammonium chloride. Cells were washed in phosphate buffered saline (PBS) and platelet contamination removed by centrifugation at 200 g for 10 min, re-suspended at a concentration of 1×10\hat{ }6 ml in RPMI-1640 culture medium. To these naltrexone was added at a concentration of either 10 uM of a conventional higher dose of naltrexone (NTX) or 10 nM low-dose naltrexone (LDN), and incubated for 48 h in a humidified atmosphere with 5% CO₂ in air at 37° C. Peripheral-blood mononuclear cells were washed twice in wash buffer (PBS containing 1% (v/v) FBS and 0.09% (v/v) NaN₃), and stained for 30 min at 4° C. with the relevant and stated antibodies for assessment of immune cell profile. Cells were washed in wash buffer and fixed in 4% paraformaldehyde for 20 min at 4° C. Expressions of the surface markers were analysed using a BD Bioscience LSR II flow cytometer with dedicated proprietary software.

Results

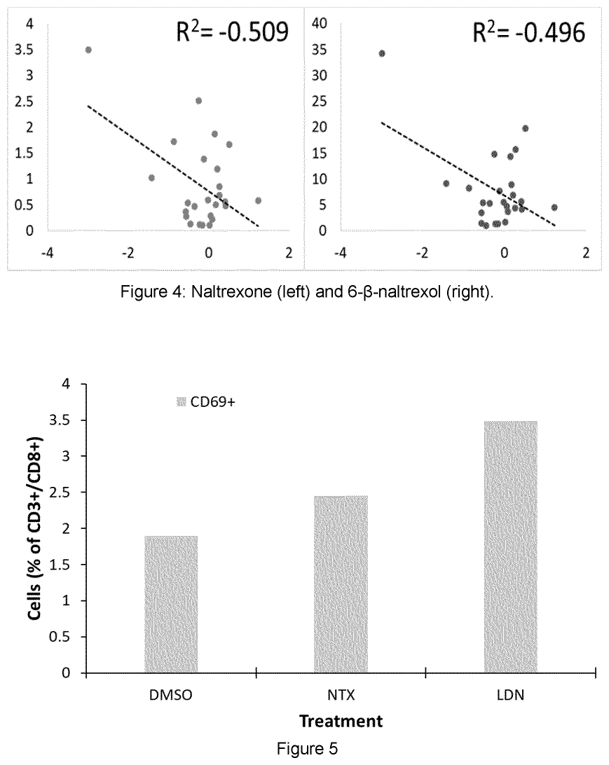

The activation state of CD3+ cells is indicated by CD69 expression; higher levels of CD69 expression correlate with increased CD3+ activation. CD3+ cells are T cell co-receptors that help to activate both the cytotoxic T cell (CD8+ naive T cells) and also T helper cells (CD4+ naive T cells). FIG. 5 shows that the CD69 expression in CD3+/CD8+ cells (killer T-cells) increased after being cultured with LDN, but after being cultureD with NTX or the vehicle control (DMSO) the CD69 expression did not increase. Thus, when LDN has completed its priming and the pERK expression is increased, the immune system is suitably boosted

1. A method for monitoring the treatment of a subject undergoing therapy with an active that is naltrexone or a metabolite or analogue thereof, comprising:
a. measuring the level of pERK in a sample obtained from the subject undergoing treatment;
b. comparing the level of pERK with a reference, wherein if the pERK level is increased compared to the reference, then the active is being administered at an effective level.

2. A method according to claim 1, wherein the reference is a sample obtained from the subject prior to treatment.

3. A method according to claim 1, wherein the sample comprises peripheral blood mononuclear cells.

4. A method according to claim 1, wherein the metabolite or analogue of naltrexone is 6-β-naltrexol, naloxone or methylnaltrexone.

5. A method according to claim 1, wherein the subject is being treated for cancer or HIV/AIDS.

6. A method according to claim 5 wherein the cancer is breast cancer, lung cancer, melanoma cancer, colon cancer, or glioma cancer.

7. A method according to claim 1, wherein if the level of pERK is at the same level or below the reference than the amount of the active is adjusted.

8. A method according to claim 1, wherein the active is administered at a level of a daily amount of no more than 4.5 mg.

9. A method according to claim 1, wherein the active is naltrexone or 6-β-naltrexol.

10. A method according to claim 1, wherein the pERK is measured at least 24 hours after administration of the active.

11. A method according to claim 9, wherein the pERK is measured at least 48 hours after administration of the active.

12. A method according to claim 1, wherein the level of pERK is determined by measuring pERK expression levels.

13. A method according to claim 1, wherein the treatment is cancer treatment.

14. A method according to claim 13, wherein the cancer treatment is chemotherapy, radiation therapy, hormonal therapy or immunotherapy.

15. A method according to claim 1, wherein the treatment is HIV/AIDS treatment.

16. A method according to claim 8, wherein the daily amount is between 3 and 4.5 mg.

17. A method according to claim 15, wherein the HIV/AIDS treatment is an antiretroviral therapy.
